(19) United States
(12) Patent Application Publication
(10) Pub. No.: US 2010/0312299 A1
(43) Pub. Date: Dec. 9, 2010

(54) CONTROL OF CARDIAC ARRHYTHMIA BY VAGAL STIMULATION AT THE ATRIOVENTRICULAR AND SINOATRIAL NODAL FAT PADS OF THE HEART

(76) Inventors: Mark Maciejewski, Edina, MN (US); Todor N. Mazgalev, Cleveland, OH (US); Youhua Zhang, BeachWood, OH (US)

Correspondence Address: TAROLLI, SUNDHEIM, COVELL & TUMMINO L.L.P., 1300 EAST NINTH STREET, SUITE 1700, CLEVELAND, OH, 44114, US

(21) Appl. No.: 12/745,115

(22) PCT Filed: Nov. 21, 2008
(86) PCT No.: PCT/US08/84275

§ 371 Date: May 27, 2010

Related U.S. Application Data
(60) Provisional application No. 60/990,375, filed on Nov. 27, 2007.

(51) Int. Cl. A61N 1/368

(52) U.S. Cl. 607/14; 607/17

(57) ABSTRACT

Vagal stimulation applied to the atrioventricular nodal (“AVN”) fat pad and the sinoatrial nodal (“SAN”) fat pad via epicardial leads is useful for controlling cardiac arrhythmia, including atrial fibrillation (‘AF”). In the case of AF, for example, vagal stimulation may be applied initially to the AVN fat pad to reduce ventricular rate, and vagal stimulation may be applied to the SAN fat pad after restoration of sinus rhythm to control atrial rate. The technique is applicable to control acute AF and chronic AF. The vagal stimulation may be optimized for exciting ganglia in the fat pads to produce dromotropic and chronotropic effects in the atrioventricular node and the sinoatrial node, respectively. In addition, the SAN fat lead can also be used to pace the atrium in case of sinus bradycardia.

### RELATED APPLICATION

This application claims priority from U.S. Provisional Application No. 60/990,375, filed Nov. 27, 2007, the subject matter of which is incorporated herein by reference.

### TECHNICAL FIELD

The present invention relates to control of cardiac arrhythmias, and more particularly to control of cardiac arrhythmias by vagal stimulation at the atrioventricular nodal fat pad and the sinoatrial nodal fat pad of the heart.

### BACKGROUND

Cardiac arrhythmias are abnormal conditions associated with the various chambers and other structures of the heart. Characterized by a rapid ventricular rate and an irregular ventricular rhythm, atrial fibrillation (“AF”) is the most frequently occurring sustained cardiac arrhythmia, particularly among the elderly, among patients with organic heart disease, and among patients recovering from coronary artery bypass graft (“CABG”) surgery; see Steinberg, Jonathan S., Postoperative Atrial Fibrillation: A Billion-Dollar Problem, Journal of the American College of Cardiology, Vol. 43, No. 6, 2004. For example, acute AF may occur in as many as 50% of patients undergoing cardiac operations. Patients with chronic AF have symptomatic tachycardia or low cardiac output and have a 5-10% risk of thromboembolic complications and events. AF is also the most common arrhythmia in clinical practice, with an overall prevalence in the general population of 0.4%.

A common treatment used to restore sinus rhythm in patients suffering from AF is cardioversion alone or in combination with anti-arrhythmic therapy, to restore sinus rhythm. Unfortunately, however, recurrence rates after such therapy can be as high as 75%. Therefore, cardioversion may not be an entirely satisfactory technique for the control of cardiac arrhythmias. When sinus rhythm restoration is not possible, ablation of the AV junction may be used to control ventricular rate, along with pacemaker implantation for controlling ventricular response. The use of anticoagulant drugs is recommended with this treatment. AV nodal ablation can, however, be disadvantageous in that the patient is rendered permanently pacemaker-dependent due to the radical destruction of the AV node.

Another common treatment is the use of rate-controlling drugs, which help control ventricular rate while allowing AF to persist. Generally suitable drugs include those classified as Ca++ blockers, digoxin, and beta-blockers. Examples of suitable antiarrhythmic drugs include amiodarone, diisopyramide, flecaimide, moricizine, procainamide, propafenone, quinidine, sotalol. These antiarrhythmic drugs may be used in conjunction with cardioversion, as necessary. The use of anticoagulant drugs is recommended with this treatment. It should be noted, however, that drugs can have limitations, such as intolerability and ineffectiveness with certain patients and incompatibility with other medications. It will thus be appreciated that treatment with rate-controlling drugs may not present an entirely satisfactory technique for the control of cardiac arrhythmias.

It has been shown that AV conduction can be slowed by selective vagal stimulation (“VS”) delivered to the so-called atrioventricular nodal fat pad; see for example, (1) Zhuang, S., Zhang, Y., Mowrey K. A., et al. Ventricular rate control by selective vagal stimulation is superior to rhythm regularization by atrioventricular nodal ablation and pacing during atrial fibrillation, Circulation, Vol. 106, No. 14, 2002, pp. 1853-1858; (2) Mazgalev, T. N., Atrioventricular node during atrial fibrillation: Is it worth saving?, J. Cardiovasc. Electrophysiol., Vol. 13, No. 7, 2002, pp. 724-726; (3) Zhang, Y., Mowrey, K. A., Zhuang, S., et al., Optimal ventricular rate slowing during atrial fibrillation by feedback AV nodal-selective vagal stimulation, Am J Physiol Heart Circ Physiol., Vol. 282, No. 3, 2002, pp. H1102-H1110; (4) Wallick, D. W., Zhang, Y., Tabata, T., et al., Selective AV Nodal Vagal Stimulation Improves Hemodynamics During Acute Atrial Fibrillation in Dogs, Am J. Physiol., Vol. 281, 2001, pp. H1490-H1497; (5) Mazgalev, T. N., Garrigue, S., Mowrey, K. A., et al., Autonomic modification of the atrioventricular node during atrial fibrillation: Role in the slowing of ventricular rate, Circulation, Vol. 99, No. 21, 1999, PP. 2806-2814; (6) Quan, K. J., Lee, J. H., et al., Identification and characterization of atrioventricular parasympathetic innervation in humans, J. Cardiovasc Electrophysiol, Vol. 13, No. 8, August 2002, pp. 735-739; and (7) Zhang, Y., Yamada, H., Bibevski, S., et al., Chronic atrioventricular nodal vagal stimulation: First evidence for long-term ventricular rate control in canine atrial fibrillation model, Circulation, Vol. 112, Nov. 8, 2005, pp. 2904-2911.

Recent experimental and clinical studies have demonstrated that selective AVN vagal stimulation can be used to slow ventricular rate during AF; however, an irregular rhythm remains. It has been proposed to combine selective AVN vagal stimulation with ventricular on-demand pacing to achieve slow, regular rhythm during AF without requiring AVN ablation. Zhang, Y. and Mazgalev, T. N., Achieving regular slow rhythm during atrial fibrillation without atrioventricular nodal ablation: Selective vagal stimulation plus ventricular pacing, Heart Rhythm, Vol. 1, No. 4, October 2004, pp. 469-475.

Despite significant advances in controlling AF without atrioventricular nodal ablation, the development of alternative techniques is desirable.

### SUMMARY OF THE INVENTION

The present invention relates to a method for controlling cardiac arrhythmia in a heart of a patient. The method includes the steps of placing a first epicardial lead at an atrioventricular nodal (‘AVN”) fat pad of the heart and placing a second epicardial lead at a sinoatrial nodal (“SAN”) fat pad of the heart. The method also includes the steps of detecting the cardiac arrhythmia and applying vagal stimulation (“VS”) to the AVN fat pad and the SAN fat pad through the first and second epicardial leads to control the detected cardiac arrhythmia.

According to one aspect of the present invention, the first and second epicardial leads may be unipolar or bipolar.

According to another aspect of the present invention, the step of placing the first epicardial lead includes the step of introducing the first epicardial lead percutaneously; and the step of placing the second epicardial lead includes the step of introducing the second epicardial lead percutaneously.

According to another aspect of the present invention, the step of placing the first epicardial lead includes the step of applying the first epicardial lead during an open heart procedure; and the step of placing the second epicardial lead includes the step of applying the second epicardial lead during the open heart procedure.

According to another aspect of the present invention, the detecting step includes the steps of acquiring an electrocardiogram of the electrical activity of the heart and identifying rate and irregularity characteristics indicative of atrial fibrillation in the electrocardiogram.

According to another aspect of the present invention, the acquiring step includes the step of applying a plurality of electrical sensing leads to skin of the patient.

According to another aspect of the present invention, the detecting step includes the steps of acquiring atrial rate information from a right atrium of the heart and identifying atrial fibrillation by the atrial rate information.

According to another aspect of the present invention, the acquiring step includes the step of introducing an electrical sensing lead into myocardial tissue of the right atrium of the heart.

According to another aspect of the present invention, the detecting step includes acquiring atrial rate information from the second epicardial lead.

According to another aspect of the present invention, the step of applying vagal stimulation includes the steps of applying vagal stimulation to the AVN fat pad through the first epicardial lead to control ventricular rate and applying vagal stimulation to the SAN fat pad through the second epicardial lead to control atrial rate.

According to another aspect of the present invention, the cardiac arrhythmia is atrial fibrillation and the step of applying AVN fat pad vagal stimulation includes the step of applying the AVN fat pad vagal stimulation initially (over the first interval) in the absence of the SAN fat pad vagal stimulation in response to the cardiac arrhythmia detecting step; and the step of applying SAN fat pad vagal stimulation includes the step of applying the SAN fat pad vagal stimulation in the absence of the AVN fat pad vagal stimulation (over a second interval).

According to another aspect of the present invention, the step of applying AVN fat pad vagal stimulation and the step of applying SAN fat pad vagal stimulation overlap.

According to another aspect of the present invention, the step of applying AVN fat pad vagal stimulation includes the step of applying the AVN fat pad vagal stimulation over a third interval between the first and second intervals, the step of applying SAN fat pad vagal stimulation includes the step of applying the SAN fat pad vagal stimulation over a fourth interval between the first and second intervals, wherein the third and fourth intervals are concurrent.

According to another aspect of the present invention, the step of applying AVN fat pad vagal stimulation includes the step of applying the AVN fat pad vagal stimulation intermittently, and the step of applying SAN fat pad vagal stimulation includes the step of applying the SAN fat pad vagal stimulation intermittently.

According to another aspect of the present invention, the method further includes the steps of establishing a first acceptable physiological level for ventricular rate, establishing a second acceptable physiological level for atrial rate, and monitoring ventricular rate and atrial rate, wherein the step of applying AVN fat pad vagal stimulation includes the steps of delivering a level of AVN fat pad vagal stimulation, determining whether the monitored ventricular rate is satisfactory relative to the first acceptable physiological level as a result of the step of delivering AVN fat pad vagal stimulation, and repeating the step of delivering AVN fat pad vagal stimulation and the step of determining the ventricular rate with the level of AVN fat pad vagal stimulation increased, if the monitored ventricular rate is not satisfactory relative to the first acceptable physiological level, and wherein the step of SAN fat pad vagal stimulation includes the steps of delivering a level of SAN fat pad vagal stimulation, determining whether the monitored atrial rate is satisfactory relative to the second acceptable physiological level as a result of the step of delivering SAN fat pad vagal stimulation, and repeating the step of delivering SAN fat pad vagal stimulation and the step of determining atrial rate with the level of SAN fat pad vagal stimulation increased, if the monitored atrial rate is not satisfactory relative to the second acceptable physiological level.

According to another aspect of the present invention, the method also includes the step of terminating the applying step if the level of vagal stimulation applied to the AVN fat pad, or the SAN fat pad, or both the AVN and SAN fat pads, reaches a predetermined maximum level of vagal stimulation.

The present invention also relates to an apparatus for controlling ventricular rate in a heart of a patient, the heart having an atrioventricular node (“AVN”), a sinoatrial node (“SAN”), an AVN fat pad containing parasympathetic ganglia that selectively innervate the AV node, and a SAN fat pad containing parasympathetic ganglia that selectively innervate the SA node. The apparatus includes a first epicardial lead for placement at the SAN fat pad and a second epicardial lead for placement at the AVN fat pad. The apparatus also includes a stimulation driver electrically coupled to the first and second epicardial leads and a source of monitoring signals indicative of atrial fibrillation. The apparatus also includes a microprocessor or microcontroller for processing the signals and controlling the stimulation driver and a memory. The memory includes instructions for monitoring for signals indicative of atrial fibrillation, detecting atrial fibrillation from the monitoring signals, applying vagal stimulation to the AVN fat pad when atrial fibrillation is detected, continuing the vagal stimulation to the AVN fat pad until atrial fibrillation ceases, and applying vagal stimulation to the SAN fat pad at least until normal sinus rhythm is attained. The apparatus applies the SAN fat pad stimulation being in the absence of the AVN fat pad stimulation at least from after atrial fibrillation ceases to when normal sinus rhythm is attained.

According to another aspect of the present invention, the monitoring signal source includes a signal input for receiving an output from a standard electrocardiograph.

According to another aspect of the present invention, the monitoring signal source includes an integrated standard electrocardiograph.

According to another aspect of the present invention, the monitoring signal source includes an electrocardiograph coupled directly to the first or second epicardial lead.

According to another aspect of the present invention, the instructions for applying vagal stimulation include instructions for delivering a level of vagal stimulation, instructions for determining whether the ventricular rate is at the acceptable physiological level as a result of the delivering instruction, and instructions for repeating the delivering instruction and the determining instruction with the level of vagal stimulation increased if the ventricular rate exceeds the acceptable physiological level.

According to another aspect of the present invention, the instructions for applying vagal stimulation further include an instruction for terminating delivery of vagal stimulation if the level of vagal stimulation reaches a predetermined maximum level of vagal stimulation.

The present invention further relates to a computer storage medium containing computer instructions for controlling ventricular rate in a heart of a patient, the heart having an atrioventricular node (“AVN”), a sinoatrial node (“SAN”), an AVN fat pad containing parasympathetic ganglia that selectively innervate the AV node, and a SAN fat pad containing parasympathetic ganglia that selectively innervate the SA node. The instructions include instructions for monitoring for signals indicative of atrial fibrillation, detecting atrial fibrillation from the monitoring signals, and applying vagal stimulation to the AVN fat pad when atrial fibrillation is detected. The instructions also include instructions for continuing the vagal stimulation to the AVN fat pad until atrial fibrillation ceases, and applying vagal stimulation to the SAN fat pad at least until normal sinus rhythm is attained, the SAN fat pad stimulation being applied in the absence of the AVN fat pad stimulation at least from after atrial fibrillation ceases to when normal sinus rhythm is attained. The instructions further include instructions for applying atrial pacing when the sinus rate is too slow after AF stops.

### DESCRIPTION OF THE DRAWINGS

FIG. 1 is a posterior perspective view of a canine heart.

FIG. 2 depicts graphs that illustrate certain experimental electrical and hemodynamic performance data associated with a heart.

FIG. 3 depicts graphs that illustrate the effect of stimulation of the SAN fat pad during sinus rhythm.

FIG. 4 is a flowchart that illustrates a technique of pacing a heart from the atrioventricular nodal fat pad and the sinoatrial nodal fat pad.

FIG. 5 is a flowchart that illustrates one technique of transitioning from atrioventricular nodal fat pad pacing to sinoatrial nodal fat pad pacing.

FIG. 6 is a flowchart that illustrates another technique of transitioning from atrioventricular nodal fat pad pacing to sinoatrial nodal fat pad pacing.

FIG. 7 is a flowchart that illustrates another technique of transitioning from atrioventricular nodal fat pad pacing to sinoatrial nodal fat pad pacing.

FIG. 8 is a posterior perspective view of a human heart.

### DESCRIPTION OF THE INVENTION

Vagal stimulation (“VS”) is applied to the atrioventricular nodal (“AVN”) fat pad and the sinoatrial nodal (“SAN”) fat pad via leads that are attached directly to the epicardium (i.e., epicardial leads) or, alternatively, that reach the fat pads via an intracardial approach (i.e., endocardial leads). Vagal stimulation is useful for controlling cardiac arrhythmia, including atrial fibrillation (“AF”). In the case of AF, for example, vagal stimulation may be applied initially to the AVN fat pad to reduce and control ventricular rate. When AF terminates and sinus rhythm resumes at a high sinus rate (sinus tachycardia), then stimulation can be applied to the SAN fat pad to slow the sinus rate to a desired level. The technique is applicable to control atrial arrhythmias, including atrial tachycardia, atrial flutter, and AF, both acutely and chronically.

Vagal stimulation may be optimized for exciting ganglia in the fat pads. The ganglionated plexus in the AVN fat pad are part of the parasympathetic nerve system and preferentially project nerve terminals to the atrioventricular node. Suitable stimulation of the vagal ganglia in the AVN fat pad produces a dromotropic effect in the atrioventricular node, which slows the ventricular rate during ongoing AF essentially by impeding the transmission of erratic impulses from the atrium to the ventricle through the atrioventricular node. The ganglionated plexus in the SAN fat pad also are part of the parasympathetic nerve system and preferentially project nerve terminals to the sinoatrial node. Suitable stimulation of the vagal ganglia in the SAN fat pad produces a chronotropic effect in the sinoatrial node, which acts to slow the sinus rate in the absence of AF.

Advantageously, the lead or leads used for the vagal stimulation also may be used with pacing stimulation to pace the atrium. The lead or leads also may be used to monitor atrial and ventricular electrical activity. Separate leads, however, could be applied to various myocardial areas of the heart to perform this function.

Also, advantageously, the treatment of cardiac arrhythmias according to the principles described herein is relatively easy to carry out, because the AVN fat pad and the SAN fat pad are readily identifiable and the ganglionated plexuses associated therewith are readily accessible via epicardial electrodes. Lead placement and, for temporary therapies, lead removal is straightforward, and does not lead to any significant scarring of cardiac tissue. The effect of neural stimulation is temporary and relatively confined to the sinoatrial and atrioventricular nodes.

### The Sinoatrial and Atrioventricular Nodes

The normal pathway for electrical activation of the heart begins at the sinoatrial (“SA”) node and extends to other regions of the heart, namely the atria, the atrioventricular (“AV”) node, the bundle of His, the Purkinje fibers, and the ventricles. Heart cells, including the heart cells in this pathway, generally are excitable cells in that they are capable of generating an electrical response known as action potentials (“APs”). The APs differ from region to region, reflecting the different roles of the different cell types.

The sinoatrial node contains specialized cells that do not have a true resting potential, but rather exhibit a slow spontaneous depolarization known as phase 4 depolarization or the pacemaker potential. These cells are spontaneously active, that is automatic, and the slope of the phase 4 depolarization is an important determinant of the rate of AP generation and heart rate. Once the phase 4 depolarization brings the cell to the threshold for AP firing, an AP occurs. Then, an upstroke (phase 0) occurs that is quite different from the upstroke in ventricular, nerve, and skeletal muscle cells, in that it is much slower and is Ca2 dependent rather than Na dependent. This slower phase 0 is significant in cardiac function because it results in a slow conduction in nodal cells.

The atrioventricular node contains specialized cells that generate APs that are similar to the APs of the cells of the sinoatrial node. The AVN cells are Ca2 dependent and display spontaneous phase 4 depolarization. However, the rate of the phase 4 depolarization in the AVN cells is much slower than the rate of the phase 4 depolarization in the SAN cells, so that the SAN cells fire APs before the AVN cells fire. The wave of depolarization from the sinoatrial node propagates to other regions of the heart, ultimately leading to contraction of the heart. Pacemaker cells in other regions of the heart such as in the atrioventricular node normally do not have an opportunity to fire spontaneously before the wave of depolarization from the sinoatrial node drives them to threshold. This is why the sinoatrial node rather than the atrioventricular node acts as the normal pacemaker of the heart.

The automaticity of the SAN cells and the AVN cells is modulated by certain neurotransmitters. Certain currents in these cells are enhanced by the sympathetic neurotransmitter norepinephrine (“NE”), and inhibited by the parasympathetic neurotransmitter acetylcholine (“ACh”). Norepinephrine increases the slope of the phase 4 depolarization so that the threshold is reached sooner and heart rate increases. Acetylcholine decreases the slope of the phase 4 depolarization so that the threshold is reached more slowly and heart rate decreases. Acetylcholine also activates another specific current that hyperpolarizes the cell, that is, drives the maximum diastolic potential further from threshold so that it takes even longer time to reach threshold. The cells of the sinoatrial node are richly innervated by sympathetic and parasympathetic nerves, so that the actions of norepinephrine and acetylcholine are greatly involved in the stimulating and inhibiting effect on heart rate of sympathetic and parasympathetic nerve stimulation.

### The Sympathetic and Parasympathetic Nervous Systems

The autonomic nervous system controls involuntary body functions. Functionally, the autonomic nervous system has two divisions, sympathetic and parasympathetic.

Sympathetic nerves act on organs and blood vessels to prepare the body to react to stressful situations by, for example, increasing the heart rate and ventricular contraction, dilating the blood vessels in skeletal muscles, constricting blood vessels in the skin and guts, increasing blood sugar level, stimulating sweating, dilating the pupils, and inhibiting activities of the guts and gastric secretion. The nerves arise mainly in the thoracic segments of the spinal cord and their axons pass through chains of ganglia on either side of the spinal column, from which they branch off to join other axons and stimulate many organs.

The parasympathetic division typically has an opposing effect to the sympathetic division. It is more active at rest, having in general anabolic effects. For example, parasympathetic nerves slow down the heart rate, constrict the pupils, and increase gastric secretion and intestinal motility. The parasympathetic nerves arise in the brain stem and the lower spinal cord, and their axons are very long. The ganglia are very near to the target organs, so that particular parasympathetic nerves typically affect only one organ.

Neural control of the heart is dependent on the levels of activity of sympathetic and parasympathetic neurons and the interactions that occur between these two limbs of the autonomic nervous system. For control of regional cardiac function, both pre-junctional and post-junctional interactions occur between the separate autonomic projections to the heart, particularly at the end-organ target sites such as the sinoatrial node, the atrioventricular node, and contractile elements of the atria and ventricles. See McGuirt, A. S., Autonomic interactions for control of atrial rate are maintained after SA nodal parasympathectomy, Am. J. Physiol. 272 (Heart Circ. Physiol. 41), 1997, H2525-H2533.

### Anatomy of the Fat Pads

Mammalian hearts have various collections of ganglia, known as ganglionated plexuses, associated with nerves. The ganglia contain many intrinsic neurons, most of which are multipolar, although some unipolar and bipolar neurons are also present. Within the ganglionated plexuses, impulses are conducted from one neuron to another at sites of functional apposition between neurons, known as synapses. Although a few synapses in the central nervous system are electrical synapses, conduction between neurons is usually by a chemical neurotransmitter released by the axon terminal of the excited or presynaptic cell. The neurotransmitter diffuses across the synaptic cleft to bind with receptors on the postsynaptic cell membrane, which effects electrical changes in the postsynaptic cell.

In the human heart, intrinsic cardiac ganglia and their associated nerves are found primarily embedded in epicardial fats, in which they form five atrial and five ventricular ganglionated plexuses. Atrial ganglionated plexuses (“AGP”) may be found on the superior surface of the right atrium (the superior right AGP), the superior surface of the left atrium (the superior left AGP), the posterior surface of the right atrium (the posterior right AGP), the posterior medial surface of the left atrium (the posteromedial left AGP) (the posterior right AGP and the posteromedial left AGP fuse medially where they extend anteriorly into the interatrial septum), and the inferior and lateral aspect of the posterior left atrium (the posterolateral left AGP). See Armour, J. Andrew, et al., Gross and microscopic anatomy of the human intrinsic cardiac nervous system, The Anatomical Record, Vol. 247, 1997, pp. 289-298. Ventricular ganglionated plexuses (“VGP”) may be found in fat surrounding the aortic root (the aortic root VGP, with right, anterior, left and posterior components), at the origins of the right and left coronary arteries, the latter extending to the origins of the left anterior descending and circumflex coronary arteries (the anterior descending VGP), at the origin of the posterior descending coronary artery (the posterior descending VGP), adjacent to the origin of the right acute marginal coronary artery (the right acute marginal VGP), and at the origin of the left obtuse marginal coronary artery (the obtuse marginal VGP). See Armour, supra. Neurons may also be located outside these sites, primarily in fat associated with branch points of other large coronary arteries.

Although the heart has many fat pads, only a few of the epicardial fat pads are distinctly identifiable and readily accessible. They are the right pulmonary (“RPV”) fat pad, which supplies nerve fibers preferentially to the superior right atrium and sinus node (SAN fat pad); the inferior vena cava-left arterial (“IVC-LA”) fat pad, which supplies nerve fibers to the AV node region (AVN fat pad) and both atria; and the superior vena cava-aorta fat pad (“SVCAC”) which provides efferent fibers to both the RPV fat pad and the IVC-LA fat pad, as well as additional fibers to both atria.

The IVC-LA fat pad, which is also known as the atrioventricular nodal (“AVN”) fat pad, is of particular but not necessarily exclusive interest for control of ventricular rate during AF because it selectively innervates the AV nodal region, and so can influence propagation of electrical activity that passes through the AV node from the atria into the ventricles. The SVC-AO is also of interest for control of AF because it provides efferent fibers to the IVC-LA fat pad and so can also influence propagation of electrical activity through the AV node.

The RPV fat pad, which is also known as the SAN fat pad, is of particular but not necessarily exclusive interest for control of sinus rate after AF because it selectively innervates the SA nodal region, and so can modulate the automaticity of the SAN cells.

Although particular emphasis herein is on the AVN fat pad and the SAN fat pad, it will be appreciated that other fat pads may be stimulated in the manner described herein to further influence propagation of electrical activity through the heart.

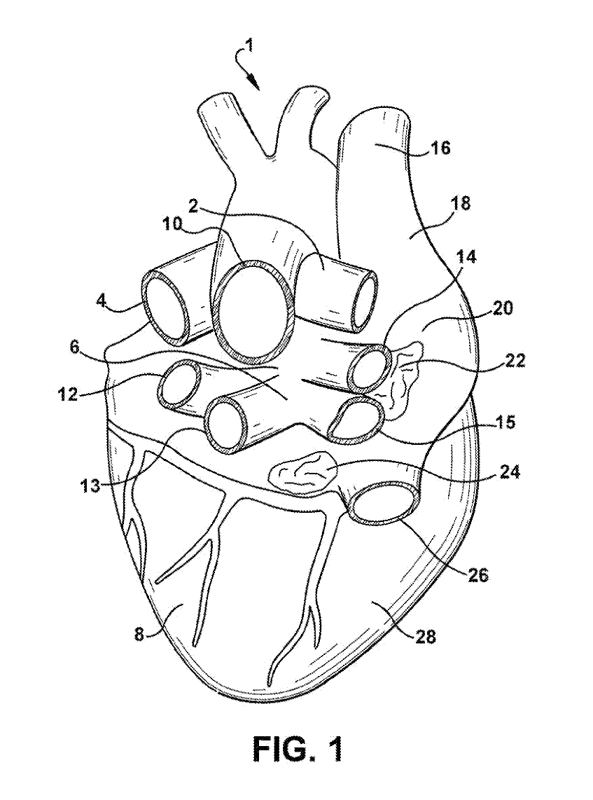

FIG. 1 illustrates a canine heart 1 upon which the systems and methods described herein were performed. Structures of interest in the canine heart 1 of FIG. 1 are: the left atrium 6, left pulmonary veins 12 and 13, right pulmonary veins 14 and 15, left ventricle 8, aorta 10, right atrium 20, high right atrium 18, superior vena cava 16, inferior vena cava 26, right ventricle 28, and pulmonary arteries 2 and 4. The AVN fat pad 24 is located at the junction of the inferior vena cava 26 and the left atrium 6. The SAN fat pad 22 is located at the junction of the right pulmonary vein 14 and the right atrium 20.

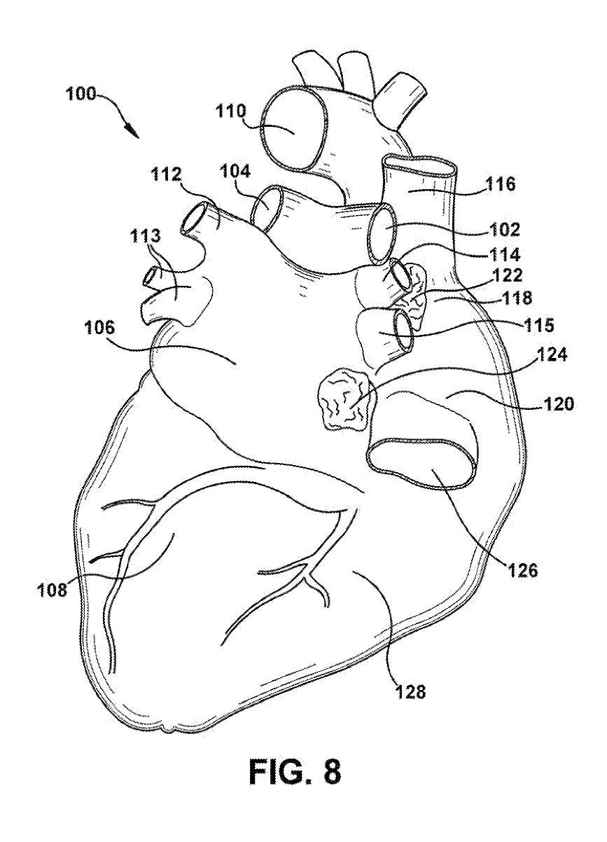

FIG. 8 illustrates a human heart 100, which is similar to the canine heart 1 of FIG. 1. Structures of interest in the human heart 100 of FIG. 8 are: the left atrium 106, left pulmonary veins 112 and 113, right pulmonary veins 114 and 115, left ventricle 108, aorta 110, right atrium 120, high right atrium 118, superior vena cava 116, inferior vena cava 126, right ventricle 128, and pulmonary arteries 102 and 104. The AVN fat pad 124 is located at the junction of the inferior vena cava 126 and the left atrium 106. The SAN fat pad 122 is located at the junction of the right pulmonary vein 114 and the right atrium 120.

### Effects of Fat Pad Stimulation on Atrial Fibrillation

Normally, the atrioventricular node plays a vital role in blocking many of the rapid atrial impulses during AF from reaching the ventricles. However, this normal filtering property of the AVN is insufficient to prevent a rapid irregular ventricular rate from being elicited during AF. Vagal stimulation of the AVN fat pad results in release of acetylcholine within the AV nodal domain, producing a negative dromotropic effect that manifests as a prolongation of the P-R interval or blockage of conduction of the AF impulses so that the ventricular rate during AF becomes substantially reduced.

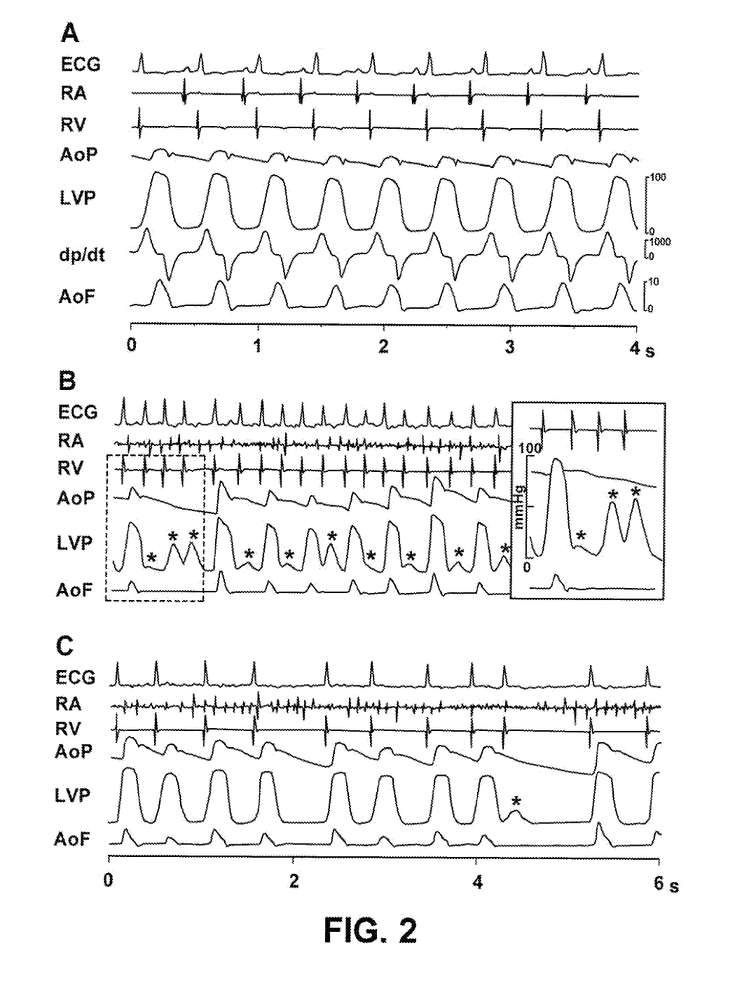

This effect may be seen in FIG. 2, which is a graph that shows certain experimental electrical and hemodynamic signals observed in a study reported in the aforementioned article by Wallick et al. Graph A shows typical electrical and hemodynamic signals during normal sinus rate, namely surface ECG, right atrial (“RA”) rate, right ventricle (“RV) rate, aortic pressure (AoP”), left ventricle pressure (“LVP”) in mmHg, first time derivative of LVP (dp/dt”) in mmHg/s, and aortic flow (“AoF”) in I/min. Graph B shows illustrative electrical and hemodynamic signals during induced AF. The significant shortened average R-R intervals during AF were accompanied with worsening of the hemodynamics. Notably, rapid irregular electrical activation of the ventricles during AF resulted in many abortive LV contractions (marked by asterisks). Graph C shows illustrative changes in the electrical and hemodynamic signals after application of AVN fat pad vagal stimulation. Notably, a dramatic reduction of the number of abortive LV contractions occurred, along with various improvements in the other hemodynamic responses.

While the AV node is primarily innervated from the AVN fat pad, it will be appreciated that other fat pads such as the superior vena cava-aorta (“SVC-AO”) fat pad may affect the release of acetylcholine within the AV nodal domain. Stimulation of such other fat pads may also be practiced to enhance the negative dromotropic effect in the AV node.

As the AF condition dissipates over time, there comes a point after which continued stimulation of the AVN fat pad after a point may be undesirable. A continuing negative dromotropic effect within the atrioventricular node can result in dropped heartbeats and blockage that can result in bradycardia even when the sinus rhythm is in excess of normal. However, cessation of AVN fat pad vagal stimulation while the sinus rate is in excess of normal may expose the heart to the stress of an unnecessarily high and sustained rate.

Additional stress to the heart is avoided by transitioning to vagal stimulation of the SAN fat pad. Stimulation of the SAN fat pad results in release of acetylcholine within the SA nodal domain. Although such a release typically would be ineffective in stopping AF, the release produces a negative chronotropic effect that is useful, when the sinus rate is in excess. Therefore, transition from AVN fat pad stimulation (when AF is present) to SAN fat pad stimulation (as AF dissipates) helps to maintain a more normal and uniform heart rate and improved hemodynamic results, without inflicting damage to either the AV node or the SA node.

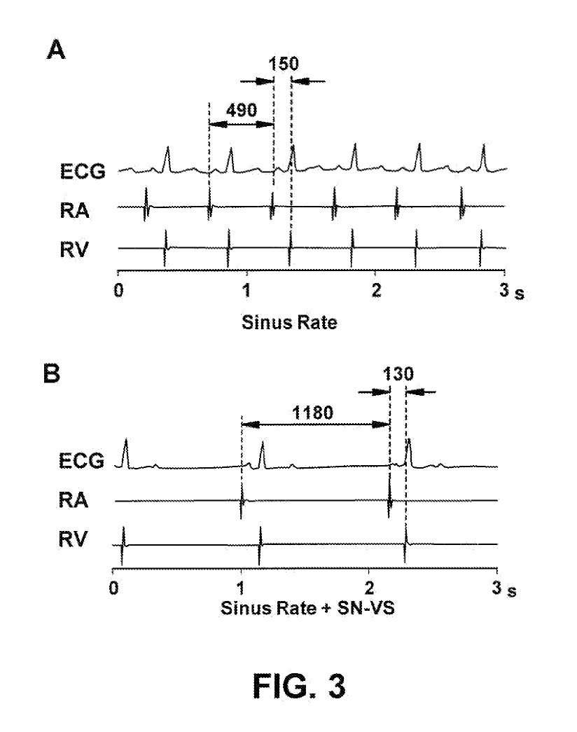

FIG. 3 shows the effect of stimulation of the SAN fat pad as observed in a study reported in the aforementioned article by Wallick et al. Graph A shows a normal sinus rhythm in which the cycle length is 490 ms and the AVN conduction time is 150 ms. Graph B shows that vagal stimulation of the SAN fat pad produced a strong chronotropic effect in that the cycle length increased to 1180 ms, and that effect was associated with a shortening of the AVN conduction time to 130 ms. Although the stimulation was performed during normal sinus rhythm, the slowing effect observed in this experiment would also occur at faster atrial rates.

Various conditions may be used to initiate a transition from AVN fat pad stimulation to SAN fat pad stimulation. A simple condition is a predetermined sinus threshold rate such as, for example, 90 beats per minute. However, any indication that the AF condition has sufficiently dissipated may be used to initiate the transition. One illustrative condition is that which occurs when AF terminates and sinus tachycardia ensues. In response to this condition, SAN fat pad stimulation can be applied to slow the sinus rate. Another illustrative condition is that which occurs when AF terminates and sinus bradycardia ensues. In response to this condition, pacing stimulation can be applied via the lead in the SAN fat pad in order to pace the atria.

Concurrent AVN fat pad vagal stimulation and SAN fat pad vagal stimulation may be used during AF. In this situation, the SAN fat pad stimulation could exert additive effect to the AVN fat pad stimulation in controlling the ventricular rate during AF. Concurrent AVN fat pad vagal stimulation and SAN fat pad vagal stimulation may be used as the AF conditions dissipates, although it may be desirable or even preferable to discontinue the AVN fat pad stimulation as the AF condition dissipates. As normal sinus rate is approached, the requisite level or degree of SAN fat pad stimulation is reduced. As normal sinus rate is achieved, the need for SAN fat pad stimulation ceases altogether. If AF reoccurs while the SAN fat pad vagal stimulation is being applied, AVN fat pad vagal stimulation is resumed.

The requisite amount or level of vagal stimulation applied to the AVN fat pad is that amount or level required to achieve a desired or acceptable (e.g., normal) heart rate during AF. The requisite amount or level of SAN fat pad vagal stimulation to apply after AF stops is that amount or level required to achieve and maintain a desired or acceptable (e.g., normal) sinus rate. These requisite amounts of vagal stimulation vary, and may be controlled by a suitable feedback system that monitors the atrial rate and the ventricular rate. Alternatively, surface ECG may be used to identify the occurrence or onset of AF and the sinus rate after AF ceases. For example, such a feedback system may work as set forth in the following paragraph.

For a particular patient, a desired heart rate is defined as a target to achieve when AF occurs. Surface ECG signals or signals from implanted leads can be used to monitor the ventricular rate, the atrial rate, or both. The system will collect and record RR interval data and will automatically calculate the actual or measured heart rate based on a defined average period, such as 5 seconds, 10 seconds, or longer. The system will compare the actual heart rate to the target heart rate, and adjust the intensity of the applied AVN fat pad vagal stimulation in response to the difference between the actual and target rates. If the actual heart rate is faster than the target heart rate, the system will increase the intensity of the AVN fat pad vagal stimulation to slow the rate. If the actual heart rate becomes slower than the target heart rate, the system will reduce the intensity of the VS to permit the heart rate to accelerate. The system will then continue monitoring the heart rate, adjusting (i.e., increasing, decreasing, or maintaining) the intensity of vagal stimulation until the actual heart rate slows to the target heart rate.

The system functions in a similar manner in applying SAN fat pad vagal stimulation. For a particular patient, a desired sinus rhythm is defined as a target to achieve when AF occurs. Upon restoration of sinus rhythm, the system determines the actual or measured sinus rhythm, compares the measured rhythm to the target rhythm, and adjusts the intensity of the applied SAN fat pad vagal stimulation in response to the difference between the actual and target rhythms. If the actual sinus rhythm is faster than the desired target, the system will increase the intensity of the SAN fat pad stimulation to slow the SAN discharges. If or when the actual sinus rhythm slows to below the desired target, the system will decrease the intensity of the SAN fat pad stimulation to allow the rhythm to increase.

Advantageously, the system can deliver the therapy described herein on-demand, with minimal latency, and can be ceased simply by terminating the nerve stimulation. The stimulation applied to the AVN fat pad and to the SAN fat pad can be varied to achieve graded ventricular rate slowing in order to produce an optimal hemodynamic response. The therapy described herein is suitable for acute AF as well as chronic AF. Additionally, in the absence of AF, the system can implement SAN fat pad stimulation to control sinus tachycardia.

Example Procedures for Controlling Heart Rate with Fat Pad Vagal Stimulation

Epicardial leads may be introduced to the atrioventricular nodal (“AVN”) fat pad of the heart and sinoatrial nodal (“SAN”) fat pad of the heart via any suitable method or technique. Examples of suitable methods or techniques for introducing epicardial leads include introducing them percutaneously using minimally invasive techniques, and introducing them directly during open heart surgery. The epicardial leads may be temporary or permanent, and may be secured using known means, such as suturing, twisting, or via a self-anchoring structure. The electrodes at the end of the leads may be of any suitable configuration or geometry, such as plate, helical, or curved, and may be of any desired polar configuration, such as unipolar, bipolar, or quadripolar. For temporary arrhythmia control, the leads may include biodegradable electrodes and other components.

Proper lead placement may be confirmed by applying stimulation and observing the electrical activity of the heart for a suitable response. For the SAN fat pad electrode, a prolongation of the PP interval, for example, may be indicative of proper lead placement at the SAN fat pad. For the AVN fat pad electrode, a prolongation of the PR interval, a skipped beat if the heart is not in AF, a slowing of the ventricular rate if the heart is in AF are all examples of responses that may be indicative of proper lead placement at the AVN fat pad.

The precise placement of a lead at the fat pad is not critical. While the lead should be placed so that the neural stimulation is directed primarily to the ganglionated plexus within the fat pad, the lead may penetrate into the myocardium of the atria for additional structural support and to permit atrial pacing. In this event, the use of separate pacing leads to the atrium is not necessary.

Any suitable technique may be used to monitor the electrical activity of the heart and detect cardiac arrhythmia, including surface electrocardiograms and right atrial and right ventricular electrograms. The right atrial and right ventricular electrograms may be taken with, for example, quadripolar plate electrodes sutured to the high right atrium 18 (FIG. 1) and right ventricular apex 28 (FIG. 1) respectively. Suitable monitoring and recording equipment is well known, and include standard units available from GE Medical Systems, a General Electric Company doing business as GE Healthcare of Waukesha, Wis. (previously Prucka Engineering, Inc.) and from CardioCommand, Inc. of Tampa, Fla. The ECG monitoring and recording system, which may be used with external standard ECG electrodes, is not used to guide or deliver therapy; instead, it permits measurement of the ventricular rate before and during delivery of AVN and SAN vagal stimulation. Alternatively, leads may be placed in other areas of the heart to monitor heart electrical activity.

A common and effective technique for monitoring for AF is to monitor the ventricular rate using a standard EGG. The use of the ventricular rate as an indicator of AF is effective because the ventricular rate during AF is irregular, and is usually more rapid than ventricular rate without AF, both without fat pad stimulation and, to a lesser degree, with fat pad stimulation. These characteristics are readily discernable from an EGG trace. Ventricular rate during AF tends to be quite high; for example, 180 beats per minute being illustrative. Application of fat pad stimulation reduces the ventricular rate; for example, 100 beats per minute being illustrative. However, the ventricular rate in both cases of AF is irregular.

While a variety of different stimulations may be applied to the AVN fat pad and the SAN fat pad to achieve a desired therapeutic effect, one type of stimulation has an amplitude, pulse width and pulse period suitable for stimulating the ganglia plexus of the parasympathetic system without capturing the ventricular myocardium nearby the atrioventricular node or the atrial myocardium nearby the sinoatrial node. An illustrative stimulation protocol of this type begins with a low stimulator output of less than about 3 mA with 50 μs pulse duration at 20 Hz. The low level stimulation is applied to the AVN fat pad, and the amplitude may be increased as necessary to slow the average heart rate down toward the normal sinus rate. It will be appreciated that although varying the amplitude is discussed in this example, other characteristics of the stimulation (pulse duration, number of pulses, frequency, etc) may be changed as well to achieve desired effects. Once the ventricular rate is slowed, the low stimulation then is applied to the SAN fat pad, and the amplitude may be increased as necessary to bring the average heart rate down to about the normal sinus rate. Stimulation is kept to a “subthreshold” level, meaning that capture of the ventricles and atria is avoided by keeping the nerve pulses at a very short duration and at a relatively low intensity. Stimulation is applied continuously or intermittently, as desired.

Application of the vagal stimulation at each amplitude level may be continuous, or may be intermittent for a suitable duration such as, for example, 1 minute, with a pause of, for example, 1 minute occurring between each application. These pauses are particular useful for determining whether or not a further delivery of neural stimulation is needed. If continuous, the vagal stimulation may be paused from time to time for determining whether or not a further delivery of neural stimulation is needed. The parameters of the vagal stimulation may be determined by the physician based on the condition of the patient. In the case of AVN-VS, for example, if the amplitude is too great, the stimulation might be pro-arrhythmic in that the atrium may be electrically stimulated by the high frequency energy (“capturing”) so that the fibrillation episode is sustained. When vagal stimulation is intended, the amplitude may be limited to a level that would not result in capturing by the atrium. The maximum amplitude may vary considerably from patient to patient, with values such as 2 mA or 3 mA being appropriate for some patients, and values of 4 mA or 6 mA being appropriate for other patients. The physician balances the risk of capture against the reduction in rate. A maximum allowable strength of stimulation may be specified. When the maximum allowable strength stimulation is reached, an alert may be generated that an uncorrectable condition has occurred so that appropriate alternative measures may be undertaken.

Neural stimulation differs from pacing the myocardium in being generally of higher frequency and lower amplitude. Pacing stimulation seeks to impose on the heart a rhythm that is compatible with normal functioning of the heart, which is generally about one hertz or so (that is, 60 or 70 beats per minute). Generally a high amplitude signal is needed to overcome the stimulation threshold of the cells in the myocardium. In contrast, a suitable signal for fat pad vagal stimulation, and in particular for AVN-VS and SAN-VS, is illustratively a sequence of rectangular pulses of less than about 0.1 ms duration, at a frequency of 20 Hz (1200 bpm) and with an adjustable amplitude of one to five mA. Higher frequencies on the order of 30 Hz and above may be used as well, and the range and steps may be varied as well.

Many suitable types of stimulators are known and are readily available. A basic simulator, for example, includes a stimulation driver circuit, and may have manually-variable stimulation parameters suitable for being operated by a trained nurse. The basic simulator should automatically provide for pauses between deliveries of the stimulation. Alternatively, a sophisticated stimulator may be used. Such a stimulator might have the capability to receive signals indicative of the electrical activity of the heart, automatically determine whether or not AF is occurring, calculate and apply the appropriate level of vagal stimulation during AF, and cease the stimulation when. AF ceases. The stimulator may receive signals indicative of the electrical activity of the heart from a standard EGG monitor; incorporate an ECG monitor connected to standard ECG electrodes, monitor heart activity directly through the epicardial fat pad leads, or monitor heart activity directly through other leads placed on or in the heart. To improve flexibility and provide for updates, the stimulator may be microprocessor or microcontroller based, and contain programmable memory. Illustrative stimulators include the Programmable Stimulator Model 5328, which is available from Medtronic, Inc. of Minneapolis, Minn.; and the Master-8 stimulator available from AMPI of Jerusalem, Israel.

The epicardial leads on the fat pats also permit the application of a pacing stimulation for capturing the atrial myocardium nearby the atrioventricular node, or the atrial myocardium nearby the sinoatrial node, or both, thereby allowing pacing of the heart in the absence of AF. Because pacing stimulation is performed at a significantly lower frequency than neural stimulation, a dromotropic effect is not produced in the AV node and ventricular beating follows atrial pacing in the normal 1:1 manner.

The transition between AVN fat pad vagal stimulation and SAN fat pad vagal stimulation should occur generally when the AF ceases, since prematurely ending the AVN fat pad vagal stimulation could lead to a reoccurrence of a dangerous rapid ventricle rate, while maintaining the AVN fat pad vagal stimulation too long (e.g., after AF terminates) could lead to irregular rhythm and skipped heart beats. Atrial rate may be monitored directly by an electrode placed in the myocardium of the right atrium. Alternatively, the AVN fat pad stimulation may be performed intermittently, and the heart rate between stimulations may be monitored by such techniques as the surface ECG to detect whether the adjusted heart rate appears relatively stable.

The transition between AVN fat pad vagal stimulation and SAN fat pad vagal stimulation may be done in different ways. In one technique, the AVN fat pad vagal stimulation is done in response to detected AF, in the absence of the SAN fat pad vagal stimulation. SAN fat pad vagal stimulation may or may not be applied along with the AVN fat pad vagal stimulation, as desired. When AF terminates, the AVN fat pad vagal stimulation is terminated and the SAN fat pad vagal stimulation is initiated if not already being applied to control sinus tachycardia. AVN fat pad vagal stimulation and SAN fat pad vagal stimulation may or may not overlap, and may or may not be spaced apart by a period of no stimulation. SAN fat pad vagal stimulation is maintained in the absence of the AVN fat pad vagal stimulation until normal sinus rhythm is achieved.

The return of normal sinus rhythm may be detected in a variety of different ways. In one technique, atrial rate may be monitored directly by an electrode placed in the myocardium of the right atrium. Alternatively, the SAN fat pad stimulation may be performed intermittently, and the heart rate between stimulations may be monitored by such techniques as the surface ECG to detect whether the adjusted heart rate appears relatively stable.

The transition between vagal stimulation and pacing may be determined as follows. When sinus bradycardia occurs after AF termination, atrial pacing through the SAN fat pad lead is necessary to maintain desired heart rate.

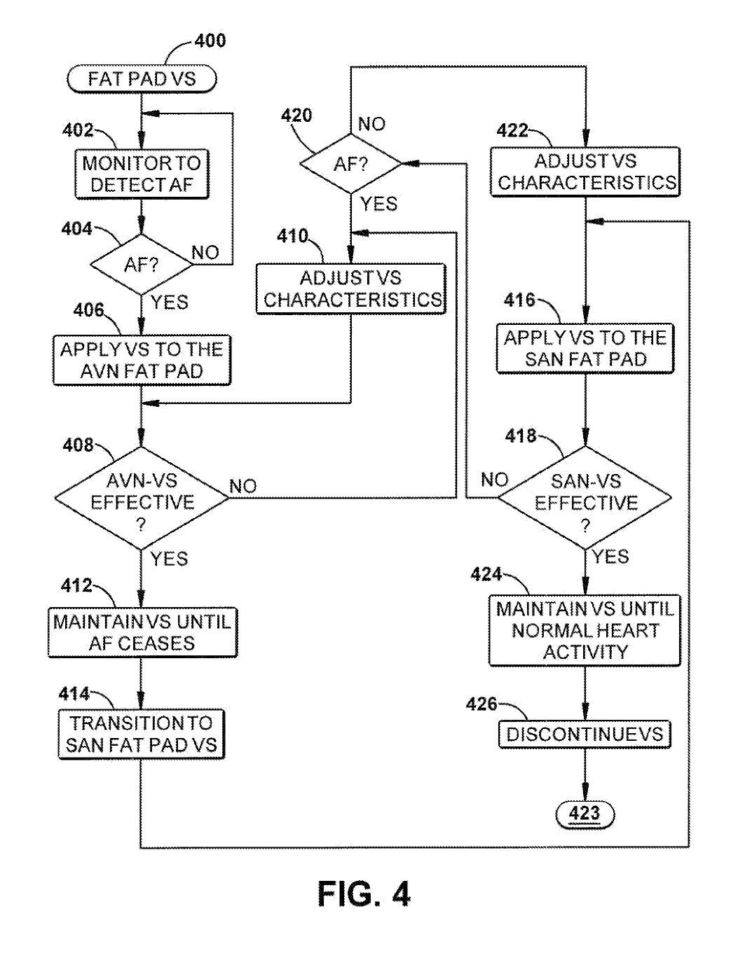

An illustrative process 400 for applying vagal stimulation is shown in FIG. 4. The heart is electrically monitored to detect AF (block 402) in any suitable manner. Such as by surface ECG or by electrodes suitably implanted in the myocardium to measure atrial rate and/or ventricular rate. When AF is detected (block 404-Y), vagal stimulation is applied (block 406) via an electrode suitably attached to the AVN fat pad to block the errant signal from the atrium. Vagal stimulation is initiated illustratively at a low amplitude such as, for AVN-VS, 1 mA. The heart rate is monitored (block 408) by monitoring the surface ECG or the ventricular rate or in any other suitable manner to detect whether the ventricular rate has been reduced to an initial acceptable physiological level, which may be a level somewhat elevated from normal but much improved over the AF levels, If the heart rate is not brought to an acceptable physiological level (block 408-N) after application of the vagal stimulation, one or more of the parameters that relate to the strength of the stimulation is gradually increased (block 410). Depending on the particular design of the neural stimulator (that is whether it has a “voltage” or “current” output), the strength of the stimuli may be increased, for example, in steps of 0.1 mA. If the physiological level is acceptable (block 408-Y), then the vagal stimulation is effective and it is maintained (with possible adjustments as necessary) until the AF ceases (block 412).

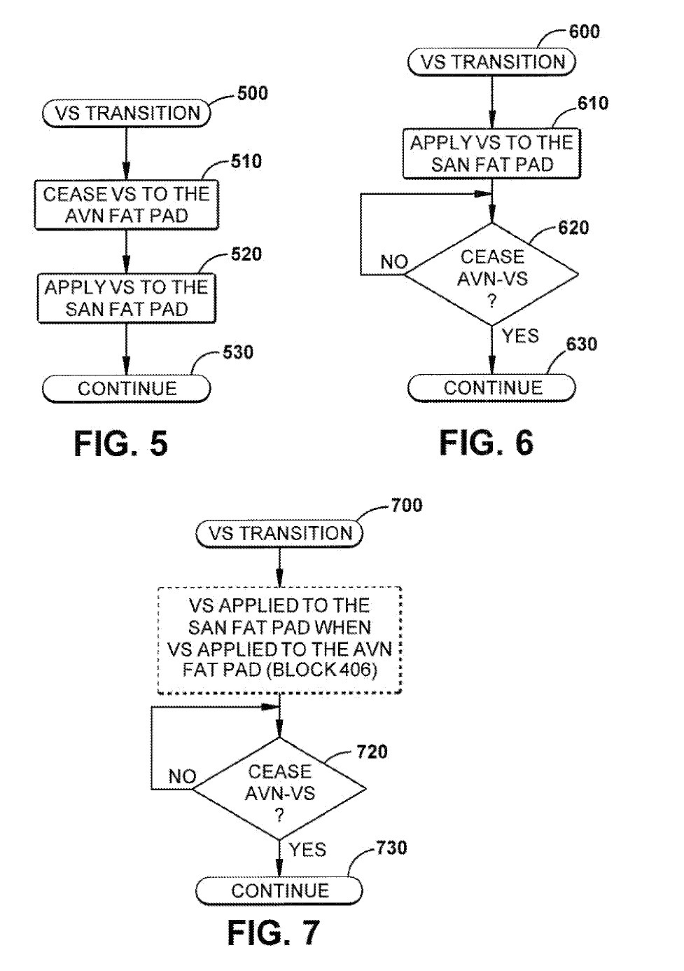

When the AF ceases and with sinus tachycardia, a transition is made to SAN vagal stimulation (block 414) by which a final acceptable heart rate may be achieved. Various different transitions may be made, and several illustrative transitions are shown in FIGS. 5-7. In the transition 500 of FIG. 5, vagal stimulation to the AVN fat pad is stopped (block 510) when AF ceases, and vagal stimulation to the SAN fat pad is applied (block 520) essentially at the same time. In the alternative transition 600 of FIG. 6, vagal stimulation to the SAN fat pad is applied as soon as AF ceases (block 610) so that vagal stimulation is simultaneously applied to the AVN fat pad and the SAN fat pad for a time. The vagal stimulation to the AVN fat pad is stopped (block 620-Y) when AF terminates. In the alternative transition 700 of FIG. 7, vagal stimulation to the SAN fat pad is presumed to have been initiated along with vagal stimulation to the AVN fat pad (in FIG. 4, block 406). The vagal stimulation to the AVN fat pad is stopped (block 620-Y) when certain conditions are established, such as the cessation of AF.

Vagal stimulation of the SAN fat pad is initiated (block 416) illustratively at a low amplitude such as, for example, 1 mA. If the heart rate is not brought to an acceptable physiological level (block 418-N) after application of the vagal stimulation, one or more of the parameters that relate to the strength of the stimulation is gradually increased and an increased vagal stimulation is applied (block 422). Depending on the particular design of the neural stimulator (that is whether it has a “voltage” or “current” output), the strength of the stimuli may be increased, for example, in steps of 0.1 mA. If the physiological level is acceptable (block 418-Y), then the vagal stimulation is effective and it is maintained (with possible adjustments as necessary) until normal heart activity is achieved (block 424). When normal heart activity is achieved, all vagal stimulation may be discontinued (block 426) and monitoring (block 402) may resume.

Low intensities of vagal stimulation may be used to obtain a moderate slowing of the ventricular rate. Alternatively, a fixed level vagal stimulation of higher intensity may be used. Generally, the particulars of the vagal stimulation set forth herein are illustrative, and beneficial effects may also be obtained with other parameter values. The effectiveness and low risk of vagal stimulation is due in part to the well recognized fact that vagal effects are directly dependent on the intensity of nerve stimulation, and that their latency is minimal. The latter means that the dromotropic effects seen in the AV node and the sinus node occur nearly simultaneous with the start of the stimulation, and also dissipate promptly at its end. These characteristic and favorable dynamics reflect the fast kinetics of acetylcholine release and hydrolysis.

The equipment for controlling arrhythmia by vagal stimulation at the AVN fat pad and SAN fat pad of the heart may be provided in kit form. One such illustrative kit includes an implantable stimulator and permanent leads for a chronic cardiac condition. Such a kit may also include sensing electrodes, which may be integrated with the stimulation electrodes or separate and distinct. Such a kit may also include a device to map the surface of the heart to determine exactly where fat pads are located.

The description of the invention including its applications and advantages as set forth herein is illustrative and is not intended to limit the scope of the inventions which is set forth in the claims. Variations and modifications of the embodiments disclosed herein are possible and practical alternatives to and equivalents of the various elements of the embodiments would be understood to those of ordinary skill in the art upon study of this patent document. These and other variations and modifications of the embodiments disclosed herein may be made without departing from the scope and spirit of the invention.

What is claimed is:

1. A method for controlling cardiac arrhythmia in a heart of a patient, the method comprising the steps of:
placing a first epicardial lead at an atrioventricular nodal (‘AVN”) fat pad of the heart;
placing a second epicardial lead at a sinoatrial nodal (“SAN”) fat pad of the heart;
detecting the cardiac arrhythmia; and
applying vagal stimulation (“VS”) to the AVN fat pad and the SAN fat pad through the first and second epicardial leads to control the detected cardiac arrhythmia.

2. The method as recited in claim 1, wherein the first and second epicardial leads are unipolar.

3. The method as recited in claim 1, wherein the first and second epicardial leads are bipolar.

4. The method as recited in claim 1, wherein:
the step of placing a first epicardial lead comprises the step of introducing the first epicardial lead percutaneously; and
the step of placing a second epicardial lead comprises the step of introducing the second epicardial lead percutaneously.

5. The method as recited in claim 1, wherein:
the step of placing a first epicardial lead includes the step of applying the first epicardial lead during an open heart procedure; and
the step of placing a second epicardial lead comprises the step of applying the second epicardial lead during the open heart procedure.

6. The method as recited in claim 1, wherein the step of detecting the cardiac arrhythmia detecting step comprises the steps of:
acquiring an electrocardiogram of the electrical activity of the heart; and
identifying rate and irregularity characteristics indicative of atrial fibrillation in the electrocardiogram.

7. The method as recited in claim 6, wherein the step of acquiring an electrocardiogram comprises the step of applying a plurality of electrical sensing leads to skin of the patient.

8. The method as recited in claim 1, wherein the step of detecting the cardiac arrhythmia comprises the steps of:
acquiring atrial rate information from a right atrium of the heart; and
identifying atrial fibrillation by the atrial rate information.

9. The method as recited in claim 8, wherein the step of acquiring atrial rate information comprises the step of introducing an electrical sensing lead into myocardial tissue of the right atrium of the heart.

10. The method as recited in claim 1, wherein the step of detecting the cardiac arrhythmia comprises the step of acquiring atrial rate information from the second epicardial lead.

11. The method as recited in claim 1, wherein the step of applying vagal stimulation comprises the steps of:
applying vagal stimulation to the AVN fat pad through the first epicardial lead to control ventricular rate; and
applying vagal stimulation to the SAN fat pad through the second epicardial lead to control atrial rate.

12. The method as recited in claim 11, wherein:
the cardiac arrhythmia is atrial fibrillation;
the step of applying AVN fat pad vagal stimulation comprises the step of applying the AVN fat pad vagal stimulation initially (over the first interval) in the absence of the SAN fat pad vagal stimulation, in response to the cardiac arrhythmia detecting step; and
the step of applying SAN fat pad vagal stimulation comprises the step of applying the SAN fat pad vagal stimulation in the absence of the AVN fat pad vagal stimulation (over a second interval).

13. The method as recited in claim 12, wherein the step of applying AVN fat pad vagal stimulation and the step of applying SAN fat pad vagal stimulation overlap.

14. The method as recited in claim 12, wherein:
the step of applying AVN fat pad vagal stimulation comprises the step of applying the AVN fat pad vagal stimulation over a third interval between the first and second intervals;
the step of applying SAN fat pad vagal stimulation comprises step of applying the SAN fat pad vagal stimulation over a fourth interval between the first and second intervals; and
the third and fourth intervals are concurrent.

15. The method as recited in claim 11, wherein:
the step of applying AVN fat pad vagal stimulation comprises the step of applying the AVN fat pad vagal stimulation intermittently; and
the step of applying SAN fat pad vagal stimulation comprises the step of applying the SAN fat pad vagal stimulation intermittently.

16. The method as recited in claim 4, further comprising the steps of:
establishing a first acceptable physiological level for ventricular rate;
establishing a second acceptable physiological level for atrial rate; and
monitoring ventricular rate and atrial rate, wherein the AVN fat pad vagal stimulation applying step comprises the steps of:
delivering a level of AVN fat pad vagal stimulation;
determining whether the monitored ventricular rate is satisfactory relative to the first acceptable physiological level as a result of the step of delivering AVN fat pad vagal stimulation; and
repeating the step of delivering AVN fat pad vagal stimulation and the step of determining ventricular rate with the level of AVN fat pad vagal stimulation increased, if the monitored ventricular rate is not satisfactory relative to the first acceptable physiological level, and wherein the step of applying SAN fat pad vagal stimulation comprises the steps of:
delivering a level of SAN fat pad vagal stimulation;
determining whether the monitored atrial rate is satisfactory relative to the second acceptable physiological level as a result of the step of delivering SAN fat pad vagal stimulation; and
repeating the step of delivering SAN fat pad vagal stimulation and the step of determining atrial rate with the level of SAN fat pad vagal stimulation increased, if the monitored atrial rate is not satisfactory relative to the second acceptable physiological level.

17. The method as recited in claim 1, further comprising the step of terminating the step of applying vagal stimulation if the level of vagal stimulation applied to the AVN fat pad, the SAN fat pad, or both the AVN and SAN fat pads, reaches a predetermined maximum level of vagal stimulation.

18. An apparatus for controlling ventricular rate in a heart of a patient, the heart having an atrioventricular node (“AVN”), a sinoatrial node (“SAN”), an AVN fat pad containing parasympathetic ganglia that selectively innervate the AV node, and a SAN fat pad containing parasympathetic ganglia that selectively innervate the SA node, the apparatus comprising:
a first epicardial lead for placement at the SAN fat pad;
a second epicardial lead for placement at the AVN fat pad;
a stimulation driver electrically coupled to the first and second epicardial leads;
a source of monitoring signals indicative of atrial fibrillation;
a microprocessor or microcontroller for processing the signals and controlling the stimulation driver; and
a memory comprising instructions for:
monitoring for signals indicative of atrial fibrillation;
detecting atrial fibrillation from the monitoring signals;
applying vagal stimulation to the AVN fat pad when atrial fibrillation is detected;
continuing the vagal stimulation to the AVN fat pad until atrial fibrillation ceases; and
applying vagal stimulation to the SAN fat pad at least until normal sinus rhythm is attained, the SAN fat pad stimulation being applied in the absence of the AVN fat pad stimulation at least from after atrial fibrillation ceases to when normal sinus rhythm is attained.

19. The apparatus as recited in claim 18, wherein the monitoring signal source comprises a signal input for receiving an output from a standard electrocardiograph.

20. The apparatus as recited in claim 18, wherein the monitoring signal source comprises an integrated standard electrocardiograph.

21. The apparatus as recited in claim 18, wherein the monitoring signal source comprises an electrocardiograph coupled directly to the first or second epicardial lead.

22. The apparatus as recited in claim 18, wherein the applying instruction comprises instructions for:
delivering a level of vagal stimulation;
determining whether the ventricular rate is at the acceptable physiological level as a result of the delivering instruction; and
repeating the delivering instruction and the determining instruction with the level of vagal stimulation increased if the ventricular rate exceeds the acceptable physiological level.

23. The apparatus as recited in claim 22, wherein the applying instruction further comprises an instruction for terminating delivery of vagal stimulation if the level of vagal stimulation reaches a predetermined maximum level of vagal stimulation.

24. A computer storage medium containing computer instructions for controlling ventricular rate in a heart of a patient, the heart having an atrioventricular node (“AVN”), a sinoatrial node (“SAN”), an AVN fat pad containing parasympathetic ganglia that selectively innervate the AV node, and a SAN fat pad containing parasympathetic ganglia that selectively innervate the SA node, the instructions comprising instructions for:
monitoring for signals indicative of atrial fibrillation;
detecting atrial fibrillation from the monitoring signals;
applying vagal stimulation to the AVN fat pad when atrial fibrillation is detected;
continuing the vagal stimulation to the AVN fat pad until atrial fibrillation ceases;
applying vagal stimulation to the SAN fat pad at least until normal sinus rhythm is attained, the SAN fat pad stimulation being applied in the absence of the AVN fat pad stimulation at least from after atrial fibrillation ceases to when normal sinus rhythm is attained; and
applying atrial pacing when the sinus rate is too slow after AF stops.
